(19) United States
(12) Patent Application Publication
(10) Pub. No.: US 2025/0017486 A1
(43) Pub. Date: Jan. 16, 2025

(54) NUCLEAR MAGNETIC RESONANCE SYSTEMS AND METHODS FOR NON-INVASIVE STIFFNESS MEASUREMENTS USING A UNILATERAL MAGNET

(71) Applicant: Livivos Inc., San Diego, CA (US)

(72) Inventor: Pablo Jose PRADO, San Diego, CA (US)

(21) Appl. No.: 18/771,910

(22) Filed: Jul. 12, 2024

Related U.S. Application Data
(60) Provisional application No. 63/526,416, filed on Jul. 12, 2023.

(51) Int. Cl. A61B 5/055; G01R 33/34; G01R 33/38

(52) U.S. Cl. CPC A61B 5/055; G01R 33/34092; G01R 33/38

(57) ABSTRACT

The present disclosure relates to nuclear magnetic resonance systems used for a non-invasive measurement of tissue in a patient. The non-invasive measurement may be conducted using a unilateral magnet.

### RELATED APPLICATIONS

This application claims the benefit of U.S. Provisional Application No. 63/526,416 filed on Jul. 12, 2023, the contents of which are incorporated herein by reference in their entirety.

### FIELD OF INVENTION

The present disclosure relates generally to the field of non-invasive evaluation and diagnostic systems and methods. More specifically, certain embodiments of the present disclosure may be directed towards non-invasive diagnostic systems and methods designed for the determination of internal organ stiffness, with some embodiments having a particular emphasis on organs such as the liver. By utilizing advanced nuclear magnetic resonance techniques and analysis algorithms, these systems and methods can enable accurate and non-invasive assessment of organ stiffness, providing valuable clinical information without the need for invasive procedures.

### BACKGROUND

Determining the stiffness of organs plays a crucial role in the diagnosis and treatment of various diseases and health conditions. Specifically, assessing liver stiffness provides valuable information, as identifying adverse changes in liver stiffness can enable early detection and treatment of diseases, resulting in improved health outcomes. The evaluation of liver stiffness aids in diagnosing and assessing risks associated with conditions such as fibrosis, cirrhosis, cancer, and other health issues. Moreover, liver stiffness, even in the absence of disease detection, serves as an important indicator of overall health.

Early monitoring of changes in liver stiffness can be a critical factor in managing various liver conditions, as may enable healthcare providers to initiate timely intervention and treatment strategies. By identifying alterations in liver stiffness at an early stage, medical professionals can implement necessary treatments before the condition progresses, potentially leading to significantly better patient outcomes. Furthermore, the accurate measurement of liver stiffness can play a pivotal role in the ongoing evaluation of how effective treatment plans are. This measurement allows for adjustments to be made based on more regularized feedback regarding the liver's response to treatment, thereby optimizing patient care. Consequently, the development of methods and devices that offer precise and reliable liver stiffness measurements is of utmost importance, as it supports both the early detection of potential liver issues and the careful monitoring of treatment effectiveness over time.

Elastography methods have emerged as valuable tools for measuring tissue stiffness, functioning as a representation of palpation-a technique used by medical providers for disease diagnosis. Palpation relies on alterations in tissue mechanics caused by conditions such as cancer, inflammation, and fibrosis.

While conventional clinical MRI modalities primarily rely on morphological changes for disease detection, elastography provides additional information beyond the capabilities of these modalities. Although Ultrasound Transient Elastography (TE) is a widely used point-of-care elastography method, its operator dependency may result in reduced accuracy. Magnetic Resonance Elastography (MRE) is the most accurate conventional method for assessing liver stiffness but is limited to clinical Magnetic Resonance Imaging (MRI) scanners, making it expensive, space-consuming, and uncomfortable for patients. Consequently, monitoring liver stiffness is not typically incorporated into routine preventative medical care, and diagnoses related to liver fibrosis are often delayed until advanced disease stages.

MRI magnets are large, expensive, and require specialized facilities with non-standard power and shielding to accommodate super-cooled magnetic coils and high magnetic fields. The cost and infrastructure demands of MRI hinder its use in preventive medicine, therapeutic efficacy evaluation, and periodic check-ups. Although MRI may assist in diagnostic and screening procedures targeting specific substances within organs (e.g., liver stiffness, fat, iron, sodium), its cost, inefficiency, and limited accessibility present significant drawbacks.

The rising incidence of metabolic syndrome underscores the need for safe, non-invasive, and cost-effective assessment tools in internal medicine and specialist practices. Discriminating patients at the highest risk of severe complications, for example type II diabetes and liver cirrhosis, has become increasingly important. These trends emphasize the necessity for periodic/regularized, accurate monitoring of clinical treatments.

Liver disease diagnostics hold particular significance. Non-alcoholic fatty liver disease (NAFLD) is the most prevalent hepatic disorder in the United States, with some NAFLD patients progressing to non-alcoholic steatohepatitis (NASH) and fibrosis, ultimately leading to cirrhosis and potentially necessitating liver transplantation. Moreover, many patients with a normal healthy weight experience various aspect of metabolic syndrome, including NAFLD, NASH, and advanced fibrosis. Delayed disease recognition in these patients compared to obese patients often leads to more severe complications. Additionally, tracking fat concentration in limb musculature serves as a valuable application due to the key findings of sarcopenia and fatty replacement in metabolically obese normal-weight (MONW) subjects.

Clinical care requires accurate assessment and grading of hepatic fibrosis severity. Quantifying liver stiffness reliably aids in grading fibrosis and longitudinally monitoring patients.

By providing accurate and affordable diagnostics for quantifying liver stiffness, early indicators can significantly reduce the incidence of liver disease.

### SUMMARY OF THE DISCLOSURE

The present disclosure relates to measuring the stiffness of organs holding significant clinical relevance, particularly in the case of the liver where stiffness directly correlates with the stage of fibrosis and increases with disease progression.

Elastography can encompass dynamic elasticity techniques that employ mechanical waves to quantitatively assess the shear modulus or stiffness of tissues. These methods rely on quantifying the velocity or wavelength of propagating shear waves within the inspected tissues, which can be induced using controlled external actuators.

The most accurate non-invasive method to measure internal organ stiffness to date is based on MRI. Clinical MRI is a well-established medical imaging technology extensively utilized in diagnostic procedures. Typically, MRI systems employ cylindrical or C-shaped magnets to create a magnetic field within an enclosure, aligning the nuclear spins of hydrogen atoms. Radio frequency (RF) pulses may then be utilized to disturb the hydrogen atom polarization, followed by the detection of the radio frequency signals generated as the atoms realign with the magnetic field. By employing space-varying magnetic fields, MRI scans a subject's body in three dimensions, generating internal images with varying contrast based on hydrogen content and tissue characteristics.

Conventional Magnetic Resonance Elastography (MRE) may employ MRI to characterize shear waves and evaluate tissue stiffness by examining the movement of mechanical waves within tissues using a specialized MRI technique. MRE is currently employed in the assessment of patients with chronic liver diseases, serving as a safe, accurate, and non-invasive alternative to liver biopsies for fibrosis staging. MRE is also being investigated for assessing other organs, including the brain, breast, blood vessels, heart, kidneys, lungs, and muscles. It represents one of the most accurate conventional, non-invasive method for detecting and staging liver fibrosis. MRE is an innovative imaging technique for non-invasive quantification of the biomechanical properties of soft tissues by visualizing propagating shear waves using modified phase-contrast MRI sequences.

To provide widespread availability of accurate fibrosis assessments, an accurate point-of-care alternative to MRE is necessary. A compact, point-of-care Nuclear Magnetic Resonance (NMR) method for quantifying liver stiffness offers a clinically valuable non-invasive means of diagnosing liver fibrosis and muscle stiffness. The concept of Point-of-Care MRE (POC MRE) introduces the accuracy of MRE in point-of-care settings. The described POC MRE concept enables stiffness measurement through a novel non-imaging NMR-based approach, eliminating the need for a full-sized MRI scanner.

POC MRE employs a compact NMR device to evaluate tissue shear modulus without generating an organ image. It utilizes shear or transverse waves, which propagate in tissue at speeds of around 1 to 10 meters per second (m/s), as opposed to longitudinal waves that travel significantly faster and cannot be accurately assessed. The dynamic properties of tissues are determined using a frequency and phase encoding method that can incorporate a constant and large magnetic field gradient generated by an open NMR probe. The POC MRE method can capitalize on the substantial field gradient produced by an open NMR probe, typically exceeding the pulse gradient strength of clinical MRI scanners by over 30 times. The POC MRE method can take advantage of the short relaxation times of water and fat in the liver at the operating field strength (lower field strength, shorter T1). With this approach, there may be a more limited or no requirement to generate an image or introduce controlled field gradients.

The results of the POC MRE technique can be directly compared with other elastography methods, as long as mechanical excitation frequencies are in similar ranges, for clinical analysis.

Embodiments of the present disclosure may include a nuclear magnetic resonance (NMR) apparatus for elastography using a constant magnetic field gradient. The NMR apparatus may include a magnetic assembly configured to generate a constant magnetic field gradient within a sample; a radiofrequency (RF) coil assembly configured to emit RF signals into the sample and receive NMR signals from the sample; a data acquisition unit connected to the RF coil assembly and configured to acquire NMR signals from the sample during elastography measurement; a processing unit connected to the data acquisition unit and configured to process the acquired NMR signals to generate elastography data based on the constant magnetic field gradient; and a display unit connected to the processing unit and configured to display generated elastography results.

Some embodiments of the NMR apparatus may also include a single-sided magnet configured to generate the constant magnetic field gradient within the sample, and an antenna connected to the RF coil assembly and configured to emit RF signals into the sample and receive NMR signals from the sample.

Some embodiments of the NMR apparatus may further include an actuator configured to transfer vibrations to a body under investigation, the actuation further including a rigid bar, and a control unit connected to the actuator and configured to control the transfer of vibrations to the body during an elastography test.

Some embodiments of the NMR apparatus may have a compact NMR device mounted on a bed or stand.

Some embodiments of the NMR apparatus may further include an external actuator positioned in proximity to an NMR probe, where said external actuator may be configured to transfer vibrations to a body under investigation.

Some embodiments of the NMR apparatus may include an external actuator positioned above a body placed on an NMR probe, where said external actuator may be configured to transfer vibrations to the body for elastography measurements.

Some embodiments of the NMR apparatus may further include an external actuator positioned on a side of a body placed on an NMR probe, where said external actuator may be configured to transfer vibrations to the body for elastography measurements.

Embodiments of the present disclosure may include a method for elastography using an NMR apparatus. Embodiments of the method may include generating a constant magnetic field gradient within a sample using a magnetic assembly; emitting RF signals into the sample and receiving NMR signals from the sample using an RF coil assembly; acquiring NMR signals from the sample during elastography measurement using a data acquisition unit; processing the acquired NMR signals using a processing unit to generate elastography signals based on the constant magnetic field gradient; and displaying generated elastography results using a display unit.

Some embodiments of the method may further include generating the constant magnetic field gradient within the sample using a single-sided magnet and emitting RF signals into the sample and receiving NMR signals from the sample using an antenna.

Some embodiments of the method may further include transferring vibrations to a body under investigation using an actuator comprising a rigid bar, wherein the actuator is controlled by a control unit.

In some embodiments of the method, the velocity of a wavelength of a shear wave may be computed by an effect of an NMR signal phase shift after a vibration is applied to a body.

In some embodiments of the method, the velocity of the wavelength of the shear wave may be computed by finding the frequencies with local minima in a phase variation of the NMR signals.

In some embodiments of the method, the velocity of the wavelength of the shear wave is computed by finding the frequencies with local minima in the phase shift of the NMR signals and estimating the wave velocity or wavelength by looking at a difference in the minima.

Some embodiments of the method may further include utilizing an external actuator positioned in proximity to an NMR probe to transfer vibrations to a body under investigation.

Some embodiments of the method may further include utilizing an external actuator positioned above a body placed on an NMR probe to transfer vibrations to the body for elastography measurements.

Some embodiments of the method may further include utilizing an external actuator positioned on a side of a body placed on an NMR probe to transfer vibrations to the body for elastography measurement.

In some embodiments of the method, a stiffness determination does not depend on a T2* measurement of the NMR signal.

Some embodiments of the method may further include a series of scans performed after short time delays and by shifting excitation frequency to avoid saturation effects, as a means of increasing a number of scans per unit of time to increase sensitivity.

In some embodiments of the method, measuring T1ρ may be deterministic of a degree of fibrosis in a body under investigation.

### BRIEF DESCRIPTION OF THE FIGURES

The technology disclosed herein, in accordance with one or more various embodiments, is described in detail with reference to the following figures. The figures are provided for purposes of illustration only and merely depict typical or example embodiments of the disclosed technology. These figures are provided to facilitate the reader's understanding of the disclosed technology and shall not be considered limiting of the breadth, scope, or applicability thereof. It should be noted that for clarity and ease of illustration, these figures are not necessarily made to scale.

FIG. 1 is a front view of an example POC MRE device, according to embodiments of the present disclosure.

FIG. 2A is a representation of an example magnetic field distribution, according to embodiments of the present disclosure.

FIG. 2B is a representation of an example gradient, according to embodiments of the present disclosure.

FIG. 2C is a representation of an example frequency distribution across the sensitive volume of the open NMR probe, according to embodiments of the present disclosure.

FIG. 3 is a representation of example time and position changes of tissue oscillation.

FIG. 4A is a representation of an example disc-shaped sensitive volume with the gradient of the magnetic field pointing across the disc, where the shape of the sensitive volume is in a plane parallel to the top of the NMR probe, according to embodiments of the present disclosure.

FIG. 4B is a representation of an example disc-shaped sensitive volume with the gradient of the magnetic field pointing across the disc, wherein the depicted thin profile is of the sensitive volume, according to embodiments of the present disclosure.

FIG. 4C is a representation of example motion of the microscopic regions in the tissue when a shear waves travels along the sensitive volume, according to embodiments of the present disclosure.

FIG. 5 depicts an example POC MRE configuration with a vibration device in proximity to the NMR probe, according to embodiments of the present disclosure.

FIG. 6 depicts an example POC MRE configuration with a vibration device in a position opposite the NMR probe, according to embodiments of the present disclosure.

FIG. 7 depicts an example POC MRE configuration with a vibration device on the side of the body, according to embodiments of the present disclosure.

FIG. 8 is a representation of an example amplitude of the shear wave in the region of the sensitive volume where example long (top) and short (bottom) wavelengths are depicted, according to embodiments of the present disclosure.

FIG. 9 is a representation of an example NMR pulse sequence where the CPMG sequence depicts an example excitation pulse and refocusing pulse, according to embodiments of the present disclosure.

FIG. 10 is a representation of an example maximum phase shift in the NMR signal attained as the vibration frequency is increased, according to embodiments of the present disclosure.

FIG. 11 is a representation of an example modulation of the phase shift at various frequencies, when interference between vibration modes is present, according to embodiments of the present disclosure.

FIG. 12 is an example diagram of a T1 rho spin-lock pulse sequence showing hard (top) and shaped (bottom) pulses (+x and −x) and the spin-lock stage, according to embodiments of the present disclosure.

FIG. 13 is a representation of wavelengths of the sensitive volume in relation to distance, according to embodiments of the present disclosure.

FIG. 14 is a representation of an example diffusion encoding pulse sequence where the excitation pulse (theta 1) and refocusing pulse (theta 2) generate a diffusion-weighted echo, followed by a train of refocusing pulses (theta 3), according to embodiments of the present disclosure.

FIG. 15 is a representation of example staggered NMR scans at varying frequencies (v), according to embodiments of the present disclosure.

FIG. 16 is a representation of an example timing of the NMR pulse sequence (depicted as an example train of pulses) started by a trigger, according to embodiments of the present disclosure.

FIG. 17 is an example position of the actuator in embodiments of the POC MRE.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION

Nuclear Magnetic Resonance (NMR) elastography represents significant advancements in medical imaging technology, particularly in the assessment of tissue stiffness and elasticity. Embodiments of the present disclosure may leverage principles of NMR to provide a non-invasive, detailed view of the mechanical properties of tissues, which can be critical in diagnosing and monitoring various diseases. As discussed, traditional methods of assessing tissue stiffness, such as palpation or ultrasound-based elastography, have limitations in terms of accessibility, accuracy, and depth of penetration. NMR elastography, however, may offer a comprehensive solution by enabling the precise measurement of tissue stiffness across different body regions, including those that are challenging to assess with conventional methods, for example, the liver. The ability to accurately characterize tissue elasticity opens new avenues in the early detection and treatment of diseases that alter the mechanical properties of tissues, such as liver fibrosis, tumours, and other pathological conditions.

Embodiments of the present disclosure introduce an innovative approach to NMR elastography, designed to enhance the accuracy, ease of use, and applicability of this imaging technique. By combining advanced NMR technology with more easily accessibly imaging apparatuses, this disclosure provides significant improvement in the measurement of tissue stiffness in the preventative medicine setting. This disclosure outlines the technical details of the apparatus and methods used in this enhanced form of NMR elastography, highlighting its potential to improve the field of medical imaging by offering a more effective means of evaluating tissue health and disease.

Embodiments of the present disclosure may provide for a nuclear magnetic resonance (NMR) apparatus, which may be configured for elastography using a constant magnetic field gradient. In the context of this disclosure, elastography may include techniques for identifying changes in the elasticity of soft tissue often resulting from various disease processes.

Embodiments of the NMR apparatus may include a magnetic assembly, which may be configured to generate a constant magnetic field gradient within a sample. The magnetic assembly may include various types of known and/or novel magnetic systems in accordance with those described herein and may be configured to generate various types of magnetic field gradients, including both constant and dynamic. The sample may be a soft tissue sample inside of a human body.

Embodiments of the NMR apparatus may include a radiofrequency (RF) coil assembly, which may be configured to emit RF signals into the sample and receive NMR signals from the sample. The RF coil assembly may include various types of known and/or novel RF coil assemblies in accordance with those described herein.

Embodiments of the NMR apparatus may include a data acquisition unit connected to one or more other features of the NMR apparatus, for example, the RF coil assembly. The data acquisition unit may be configured to acquire NMR signals from the sample during elastography measurement. The data acquisition unit may include various sensors, receivers, and monitoring systems in accordance with those described herein.

Embodiments of the NMR apparatus may include a processing unit. The processing unit may be a non-transitory computer/machine readable medium with instructions that are executable thereon. The processing unit may be connected via wired or wireless methods to the data acquisition unit and may be configured to process the acquired NMR signals to generate elastography data based on the constant or dynamic field gradient.

Embodiments of the NMR apparatus may include a display unit. The display unit may be connected to the NMR apparatus via wired or wireless methods and may be integrated into the NMR apparatus or wireless communicated with at a separate location (e.g., at a computer monitor several meters away from the NMR apparatus). The display unit may be connected to the processing unit and may be configured to display generated elastography results.

### Open NMR Elastography Principle

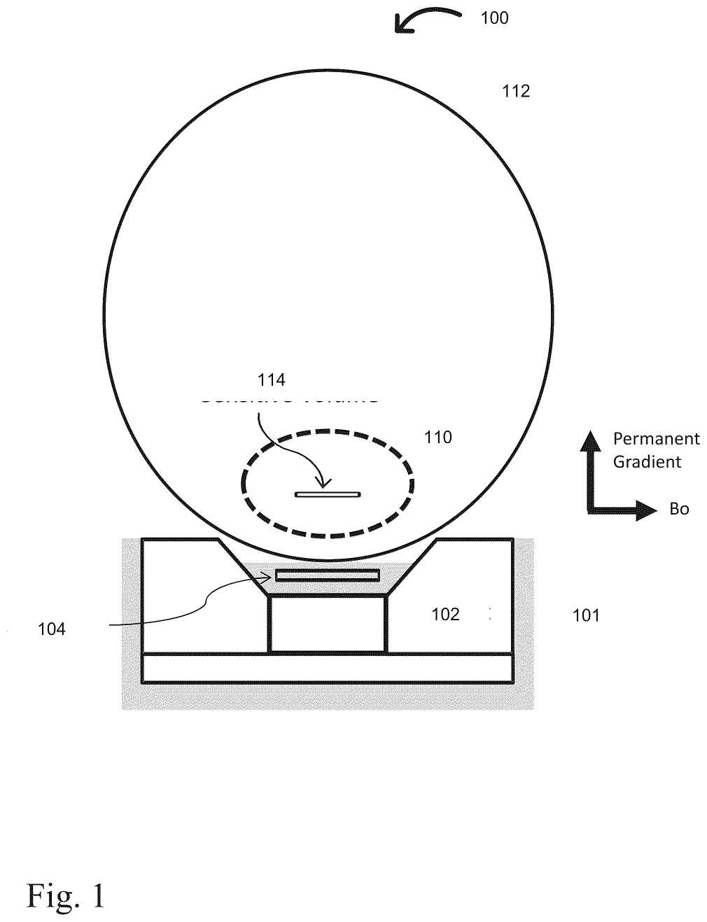

Embodiments of the present disclosure may utilize an open NMR elastography principle. Some embodiments, for example FIG. 1, may use a single-sided magnet configured to generate a constant or dynamic magnetic field gradient within a sample and may further include an antenna connected to an RF coil assembly and configured to emit RF signals into the sample and receive NMR signals from the sample. With respect to figures, FIG. 1 is a front view of an example POC MRE device 100, according to embodiments of the present disclosure. The POC MRE device 100 may have an open NMR probe system 101 that may further include a magnet 102 (which may be a single-sided magnet) and an antenna 104, which may be directed towards the organ 110 of a patient's body 112. The POC MRE device 100 may facilitate the examination of various organs, including the liver and muscles.

Sensitive Volume 114 represents the section from which the NMR signals are collected.

The POC MRE device 100 described herein may be based on a device generally or specifically designed to measure tissue properties in vivo, utilizing an open concept NMR device instead of a clinical MRI scanner. The POC MRE device 100 can incorporate open magnets 102 and antennas 104, which may enable the interrogation of tissues within the body 112, as illustrated in FIG. 1. Unlike traditional MRI, there is generally no requirement for the individual to enter the confined space of a magnet tunnel, but instead stand, sit, or lay on a medical table or similar fixture while imaging takes place. Further embodiments may include mounting the POC MRE device 100 on a bed or stand such that the patient may be able to lay, sit, or stand in proximity to the mounted device 100.

The open NMR device discussed herein can generate images but does not necessitate the generation of images. Instead, the disclosed POC MRE device 100 can leverage the advantages of MRI's non-invasive analysis to provide more accurate point-of-care measurements in a rapid and cost-effective manner by relating measurements taken by the POC MRE device 100 to muscle or tissue stiffness without the need to generate observable images. Such measurements may be observed using open NMR devices, for example the discussed POC MRE device 100, have already demonstrated the capability to measure various tissue properties, including liver fat, NMR T1 and T2 values, and diffusion coefficients.

However, with the integration of a controlled shear-wave generation attachment, as discussed herein, the present POC MRE device 100 may improve upon prior devices by becoming capable of measuring mechanical properties of tissues, specifically tissue stiffness. These measurements can be performed by assessing tissue elasticity through the detection of mechanical responses to external vibrations.

Elastography methods, such as those disclosed herein, may utilize the propagation of transverse or shear waves within liver tissue to probe its stiffness. In NMR-based methods, the movement of protons within the tissue can be examined by the effect of magnetic field gradients on the phase of the NMR signal during tissue displacement. The movement of protons within a non-uniform field can alter their resonance frequency, which may be proportional to the magnetic field strength. This effect can be measured by observing changes in the phase of the NMR signal (a frequency change over a predetermined time). To more accurately measure tissue movement caused by the small displacements generated during elastography's mechanical vibrations, on the order of microns, the effect can be amplified by employing large field gradients.

In conventional MRE, which often utilizes an MRI scanner and a mechanical actuator, a controlled field gradient can be generated using a set of gradient coils. In POC MRE, the field gradient can be constant and strong at the NMR-sensitive region that is positioned at the fringe of an open magnet. Since the effect of shear waves on NMR signals, such as phase shifts, may be influenced by the magnetic field gradient strength, a device with robust gradients offers a highly effective manner to probe and amplify tissue responses to mechanical stimulation.

One clinical application of NMR-based elastography is the non-invasive assessment of hepatic fibrosis, which is associated with an increase in liver tissue stiffness. Similarly, this method can be utilized for other tissues, such as muscle tissue.

POC MRE can utilize dynamic wave propagation techniques, which share fundamental principles with both MRE and Ultrasound TE. This can enable the measurement of a tissue's shear modulus by analyzing the effects of shear wave propagation and utilizing appropriate wave equations. The unique aspect of the POC MRE method lies in the characteristics of the field distribution, such as a constant gradient, and the evaluation of the response of an NMR signal, without the need to render an image. However, other embodiments may employ a dynamic field gradient.

The main differentiators between MRE and POC MRE are:
(1) POC MRE can use a non-imaging technique that quantifies the effect of shear waves on the partial or overall nuclear spin systems within a preselected sensitive volume.
(2) POC MRE can harness the intrinsic permanent magnet of the open magnet probe, eliminating the need for polarity-switching field gradients as used in conventional MRE. All or most relative phase changes are controlled by the pulse sequence's phase and timing, rather than a switchable gradient.
(3) POC MRE can utilize a high field gradient, typically on the order of 2 T/m, which is significantly larger than those generated by clinical MRI scanners, commonly exceeding 30 times in magnitude. This exceptional field gradient enables a highly sensitive detection of tissue stiffness.
(4) Actuators can be placed directly on the body or in the proximity of the NMR probe, without concerns about exposure to high magnetic fields, thanks to the limited extension of the open NMR device. In MRI, all metallic components of the actuator must be positioned at a distance from the scanner.
(5) POC MRE minimizes the influence of iron overload as the pulse sequences are not based on T2* measurements. This reduction in confounding effects enhances the accuracy of tissue stiffness assessments.

The main steps for POC MRE may include:
(1) Inducing shear waves in the tissue using an external driver generating mechanical vibrations on the body. The direction of shear wave propagation may be mainly or substantially parallel to the plane of the sensitive volume, allowing for tissue motion in the direction of the strong field gradient
(2) Measuring NMR signal phase shifts at given vibration frequencies by using a dedicated NMR technique (not based on any addition of magnetic field gradient, like in MRE).
(3) Determining the phase shifts modulation with frequency and using this information to compute the shear wave velocity and wavelength.

The magnetic field gradient employed in POC MRE may be high, for example in the order of 2 T/m, surpassing the gradient typically achievable by clinical MRI scanners, which is typically below 0.05 T/m. This significant difference in field gradient magnitude enables POC MRE to exhibit remarkable sensitivity in detecting shear waves and quantifying elastic effects.

As the resonance frequency (v) in NMR may be proportional to the magnetic field strength (B), and related as such: v=γB.

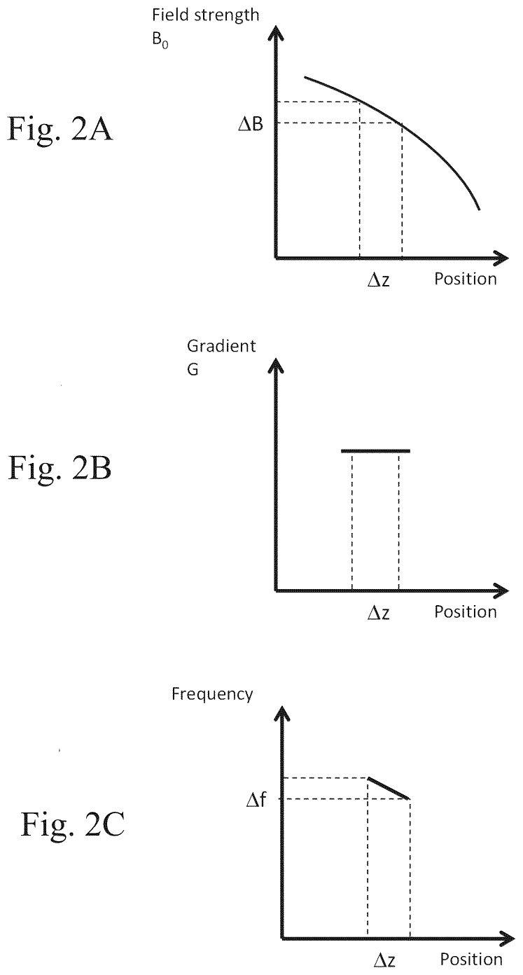

The extent of the sensitive volume (region interrogated by the open NMR probe) may be determined by the field distribution and the frequencies excited by the NMR RF pulses. Given the high gradient of the open NMR device, the thickness of the excited region can be hundreds of microns, defined by the frequency range corresponding to the NMR RF pulse duration (Fourier transformation of the pulse). With a custom magnet and antenna design, this gradient may be effectively constant over the sensitive volume, for example, as shown in FIG. 2.

FIG. 2A is a representation of an example magnetic field distribution where change in field strength ΔB is compared to position Δz, according to embodiments of the present disclosure. FIG. 2B is a representation of an example gradient where the gradient G is compared to position Δz, according to embodiments of the present disclosure. FIG. 2C is a representation of an example frequency distribution across the sensitive volume of the open NMR probe where frequency Δf is compared to position Δz, according to embodiments of the present disclosure.

As such, the sensitivity of POC MRE can arise from the interplay between phase changes in the NMR signal, the field gradient, and the intensity or amplitude of the shear waves. As a result, POC MRE is capable of acquiring probing data with phase shifts in significantly shorter time scales, when compared to conventional MRE.

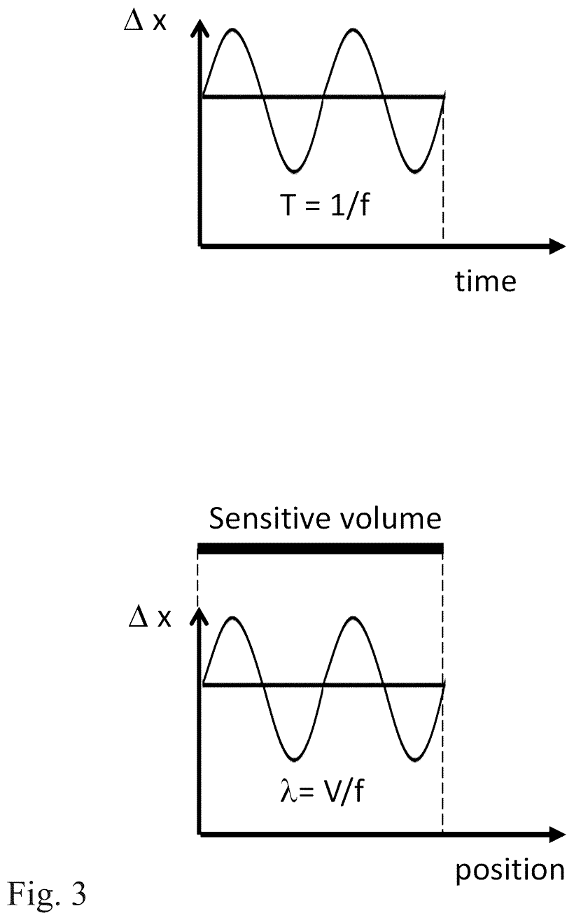

The frequency of the waves can be dictated by the external vibration, while the wavelength and velocity may be dictated by the medium in which the shear wave propagates. For example, as depicted in FIG. 3 showing an example of time and position changes of the tissue oscillation Δx (where the time evolution of the shear wave [top] has a period, T, dictated by the vibration frequency, and the wavelength [bottom] is determined by the velocity of the shear wave propagation).

### Generating Shear Waves in Open NMR

Embodiments of the present disclosure may incorporate a shear wave generator or actuator, which may be an external device responsible for applying mechanical vibrations or oscillations to the body. The actuator may be specifically designed to generate mechanical waves capable of propagating through the body and inducing small displacements in the tissues, typically on the order of a micron.

Mechanical excitations may be produced by actuator devices, including pneumatic, electromechanical, and piezoelectric systems. The wave generation mechanism may or may not be synchronized with the NMR pulse sequence. These devices can generate waves at the skin surface. These vibrations then may travel into the body and undergo mode conversion at internal tissue interfaces, ultimately giving rise to transverse shear waves.

The present POC MRE device may exhibit a constant and strong field gradient across the sensitive volume. To detect the shear waves, the oscillation may involve tissue movement parallel to the direction of the field gradient, as this condition induces phase shifts in the NMR signals. This may impose specific conditions on the generation of vibrations and the collection of signals.

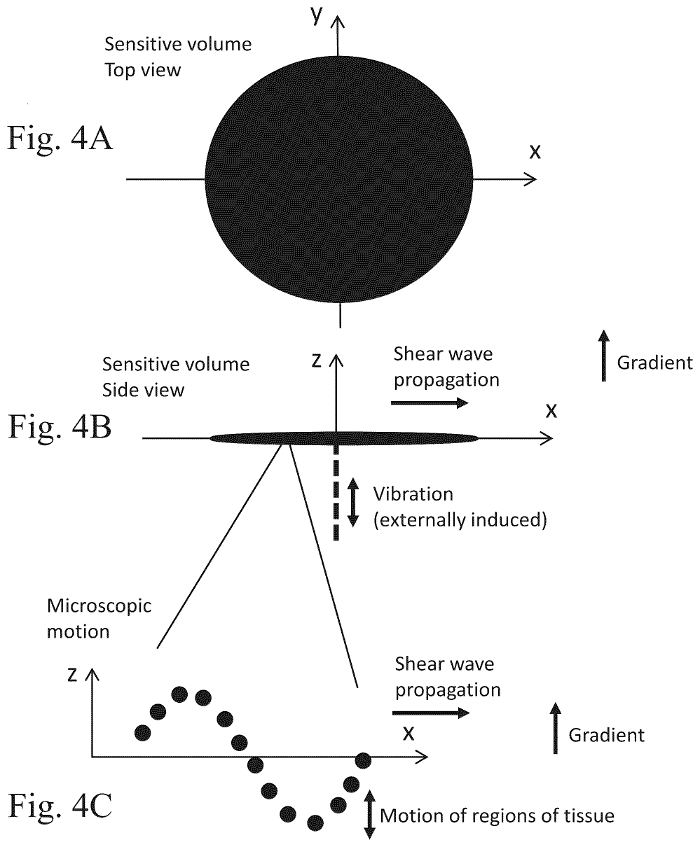

In an example of the present open NMR device, the sensitive volume may take the form of a thin disc or bowl-shaped region. Within this volume, the shear waves may propagate and the protons in the tissue may be exposed to different field strengths, resulting in NMR signal dephasing, as represented in FIG. 4.

FIG. 4A is a representation of an example disc-shaped sensitive volume with the gradient of the magnetic field pointing across the disc, where the shape of the sensitive volume is in a plane parallel to the top of the NMR probe, according to embodiments of the present disclosure. FIG. 4B is a representation of an example disc-shaped sensitive volume with the gradient of the magnetic field pointing across the disc, wherein the depicted thin profile is of the sensitive volume, according to embodiments of the present disclosure. FIG. 4C is a representation of example motion of the microscopic regions in the tissue when a shear waves travels along the sensitive volume, according to embodiments of the present disclosure. The thickness of the sensitive volume may be primarily determined by the strength of the magnetic field gradient and the bandwidth of the NMR RF pulses.

The following section presents distinct example configurations for shear wave generation within the sensitive volume. These examples serve to illustrate the general direction of vibration wave travel; however, it should be noted that the transfer of vibration modes to shear waves occurs in intricate ways, influenced by the presence of non-uniform tissue along the oscillation path.

The displacement of tissues induced by shear waves typically ranges on the order of a micron, while the sensitive volume of the POC MRE device, as demonstrated here, possesses a width spanning hundreds of microns. This substantial difference in scale highlights the high sensitivity and precision of the device in capturing tissue dynamics.

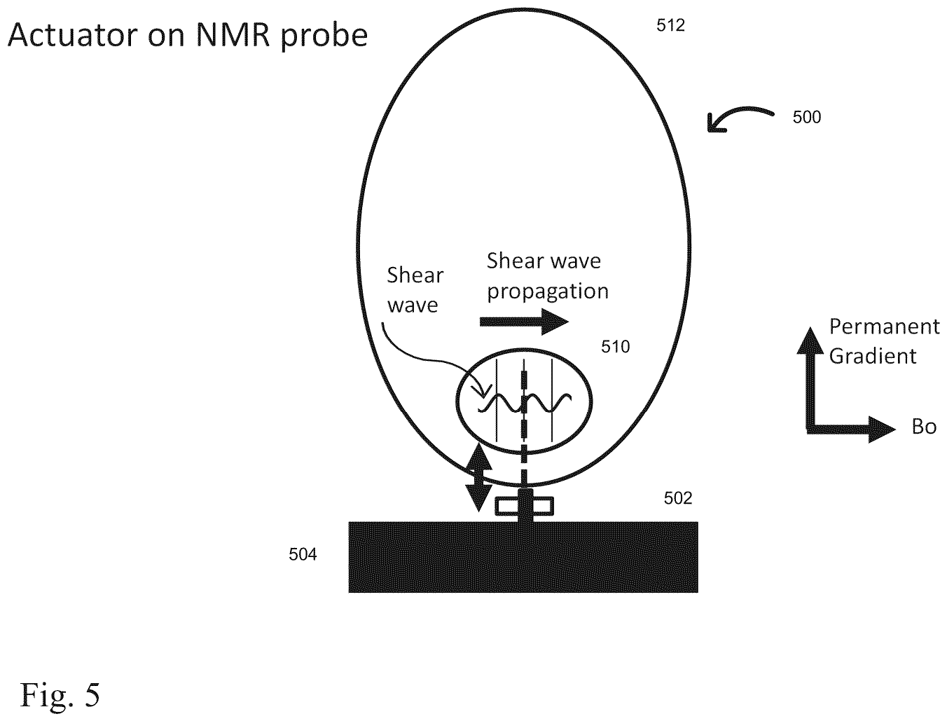

The configurations presented below provide examples of actuators capable of generating waves by applying vibrations at specific points or areas on the body. This approach shares similarities with the methodology employed in TE, further underscoring the compatibility and familiarity of the proposed technique within the broader field. FIG. 5 depicts an example open NMR device 500 with vibrations generated by an actuator device 502 on the NMR probe 504 directed at a body 512 containing an organ 510.

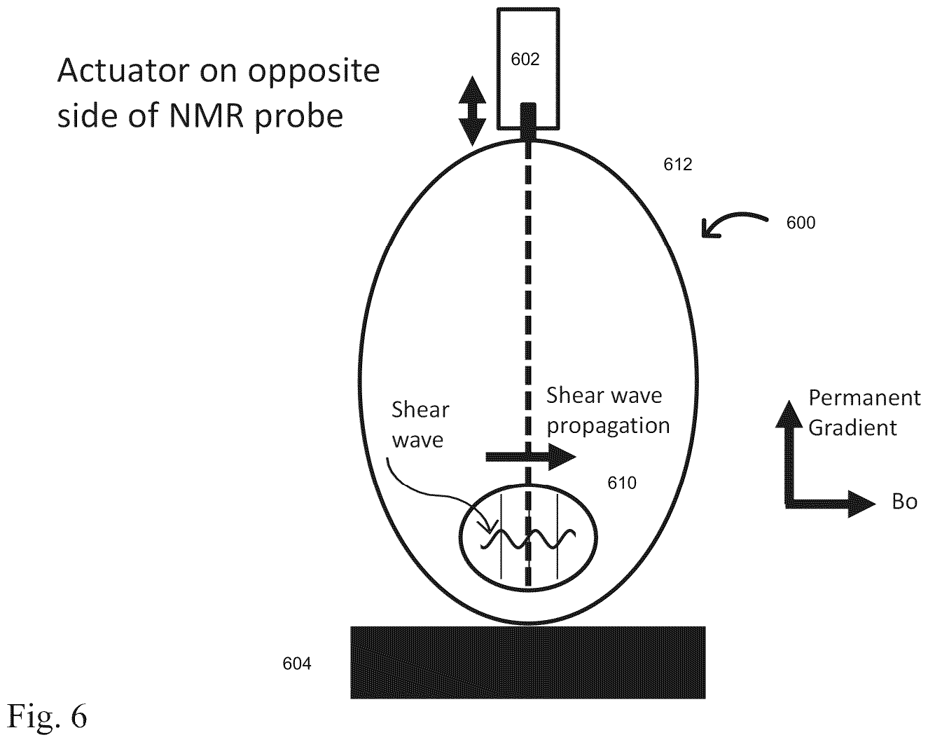

The actuator device 502 can be positioned beneath the magnet on the NMR probe 504, utilizing a non-magnetic rod that traverses through the center of the RF coil (not shown). Alternatively, it can be implemented as a slender vibrating element placed between the body and the magnet cover, as, for example, depicted in FIG. 6. FIG. 6 depicts an example POC MRE device 600 with a vibration device 602 in a position opposite the NMR probe 604 directed at an organ 610 in a body 612, according to embodiments of the present disclosure. In such an example, the shear waves may travel perpendicular to the axis of the stimulus vibration.

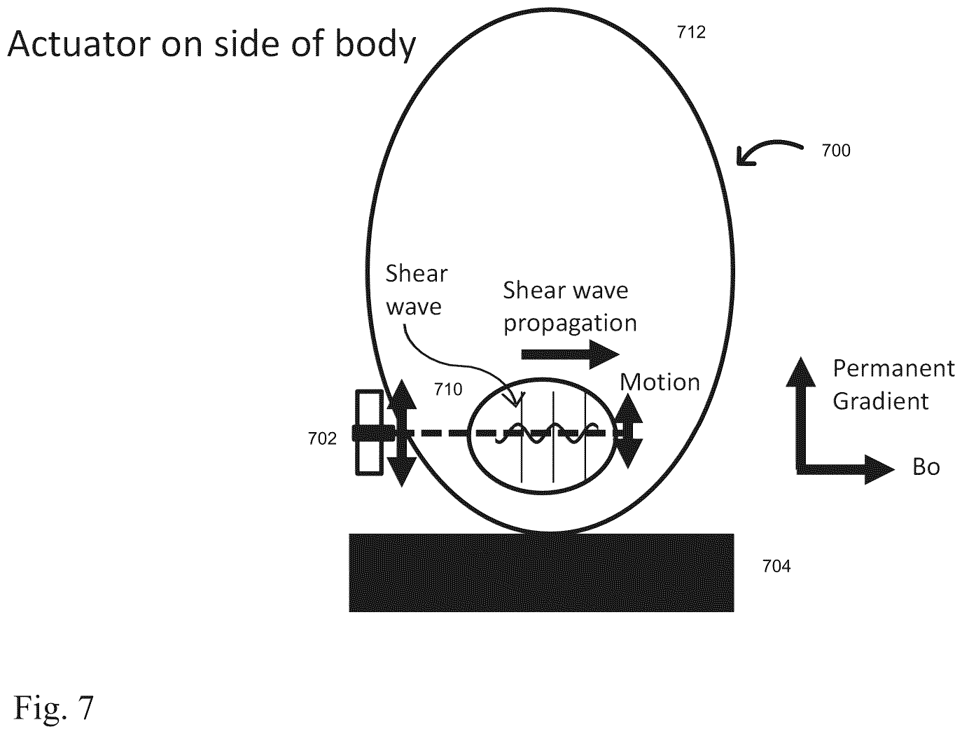

Another example configuration may have an actuator generating waves by a vibration parallel to the body, as shown in FIG. 7. FIG. 7 depicts an example POC MRE device 700 with a vibration device 702 on the side of the body 712 (approximately perpendicular to the open NMR probe 704) directed at an organ 710, according to embodiments of the present disclosure. In such an arrangement, the amplitude of the shear wave may exhibit spatial variations throughout the sensitive volume due to damping effects during its propagation deeper into the body.

Following the application of continuous harmonic motion to the tissue, a precisely synchronized NMR pulse sequence may be employed to assess the impact of the wave on the nuclear spins situated within the sensitive volume. A specialized pulse sequence can be utilized to render the signal responsive to phase fluctuations arising from the mechanical vibrations.

### Stiffness and Shear Modulus

The slope of the stress-strain curve can represent the “elastic modulus,” a fundamental parameter that can characterize the stiffness of a tissue. In order to determine the elastic modulus, shear waves (transverse waves) may be induced within the body. While purely (or near purely) elastic materials exhibit linear stress-strain behavior in accordance with Hooke's Law, many biological tissues demonstrate viscoelastic properties, combining both elastic and viscous characteristics. In such tissues, the elastic modulus can become a complex entity comprising a storage modulus (representing elasticity) and a loss modulus (representing viscosity). The viscosity component can exhibit a damping effect, where strain rate varies with time and can be characterized by a hysteresis loop resulting from energy losses during loading and unloading of stresses. Mathematical models, including those proposed by Maxwell, Voigt, and Kelvin, have been developed to predict the stress-strain dynamics of viscoelastic materials.

Depending on the nature of the stress encountered, the elastic modulus may commonly be reported as Young's modulus, E {[longitudinal (compressive or tensile) stress]/longitudinal strain}, shear modulus, μ{[shear (transverse) stress]/shear strain}, or bulk modulus, K (volumetric stress/volumetric strain). Elasticity measuring techniques typically assume that tissues are linearly elastic, isotropic, and Hookean. The elastic properties assessed through palpation are expressed as Young's modulus (E) or shear modulus (μ). For most soft tissues, Young's modulus and shear modulus are related by a simple scale factor: E=3μ, meaning that the calculation of Young's modulus or shear modulus may provide the same information. Furthermore, the bulk modulus (K) may not significantly vary in biological tissues, thus the focus of the POC MRE method may be on the elastic modulus in certain embodiments.

Under the assumption of incompressibility, the shear wave speed (Vs) and the elastic modulus (E) may be related by the following equation: E=3μ=3ρVs\hat{ }2, where ρ represents the tissue density. The wave speed can be expressed as the product of the operating frequency and the spatial wavelength. Therefore, E=3ρλ\hat{ }2f\hat{ }2, where λ denotes the wavelength of the shear wave and f represents its frequency.

A point to note is that determining the velocity of the shear wave may be sufficient to assess tissue stiffness, as the frequency of oscillation can be determined by the external stimulus. The stiffness may be directly proportional to the shear modulus and may be measured in kilopascals (kPa). To convert the shear modulus into kilopascals, the following formula may be employed: liver stiffness (kPa)=μ/1,000. For example, a shear wave speed of 2 m/s in a medium with a density of 1,000 kg/m\hat{ }3 yields μ=1,000 kg/m\hat{ }3×4 m\hat{ }2/s\hat{ }2=4,000 kg/(ms\hat{ }2). Therefore, in this example, the stiffness of the liver would be 4 kPa.

Based on MRE results and receiver operating characteristic (ROC) analysis, a cutoff value of 2.93 kPa may be used as an optimal threshold for distinguishing healthy livers from fibrotic ones, exhibiting sensitivity and specificity values of 98% and 99%, respectively.

### Shear Waves in Permanent Field Gradients

Shear wave motion in tissues may induce cyclic spin displacement of protons, which in the presence of the strong gradient of POC MRE, may result in phase shifts in the NMR signal. From this effect, the shear wave velocity can be computed. This information may then be used to quantify the tissue stiffness.

The shear waves utilized in POC MRE may fall within the audio frequency range of 10-500 Hz and are generated by pulsations applied to the skin. In the presence of a magnetic field gradient, the motion of nuclear spins may induce a phase shift (φ) in the NMR signal, as described by the equation:

φ=γ∫₀_{^{−}}⁻G(t)·r(t)dt, where γ represents the gyromagnetic ratio of the nuclei, τ is the duration of the gradients after excitation, G(t) is the magnetic gradient (which remains constant in POC MRE), and r(t) describes the position of the nuclear spins as a function of time.

For optimal sensitivity to stiffness measurements, the direction of displacements caused by the shear wave and the gradient may be parallel to maximize the product Gε, where ε represents the displacement vector. In this case, the phase shift (φ) may be on the order of Gε₀t, where ε₀ is the peak displacement.

Due to the high strength of the gradient in POC MRE, the phase shift can be detected within a single cycle. However, techniques to further enhance sensitivity by repeating the test can also be implemented.

A series of NMR signals can be collected with varying delays between the excitation pulse, the mechanical wave, and/or by switching the phase of the NMR pulses to eliminate non-motion-related phase information. Altering these time delays in subsequent acquisitions may allow for the acquisition of snapshots illustrating the propagation of the waves. By analyzing the NMR responses to the tissue dynamics induced by the shear wave propagation, the local wave speed can be computed, providing valuable information on tissue stiffness.

### Shear Wave Sensing Methods

### Controlled Vibration and Shear Waves

In present embodiments of POC MRE, the field gradient may be exceptionally strong, resulting in a free induction decay time (T2*) on the order of microseconds, which can make it challenging or impossible to read NMR signals unless special techniques are used. To overcome this, it may be a practice to acquire the NMR signal using refocusing echoes, which exhibit an effective decay time (T2) in the range of milliseconds to hundreds of milliseconds. This can enable spin-echo signal measurement and signal averaging by acquiring trains of echoes with echo separations of hundreds of microseconds.

### Shear Waves in Open NMR

In open NMR devices the size of the sensitive volume may be dominated by the bandwidth of the RF pulses, as the field gradient is high. For example, in a device with G=2 T/m, the sensitive volume may be extended over a region scaled by the size of the magnet and the antenna, but the thickness of the sensitive volume may be in the range of hundreds of microns. This is because the excitation and refocusing pulses may have a bandwidth typically of hundreds of kHz.

The shear waves may introduce displacement of tens of microns, which is often much smaller than the thickness of the sensitive volume. Therefore, dedicated methods to provide NMR sensitive to the shear waves are necessary. The disclosed methods may increase sensitivity by reducing the excited and/or observed bandwidth (excite and/or refocus spins in a narrow frequency range) or by using pulse sequences that introduce sensitivity to movement at the frequency of the vibration or close to it.

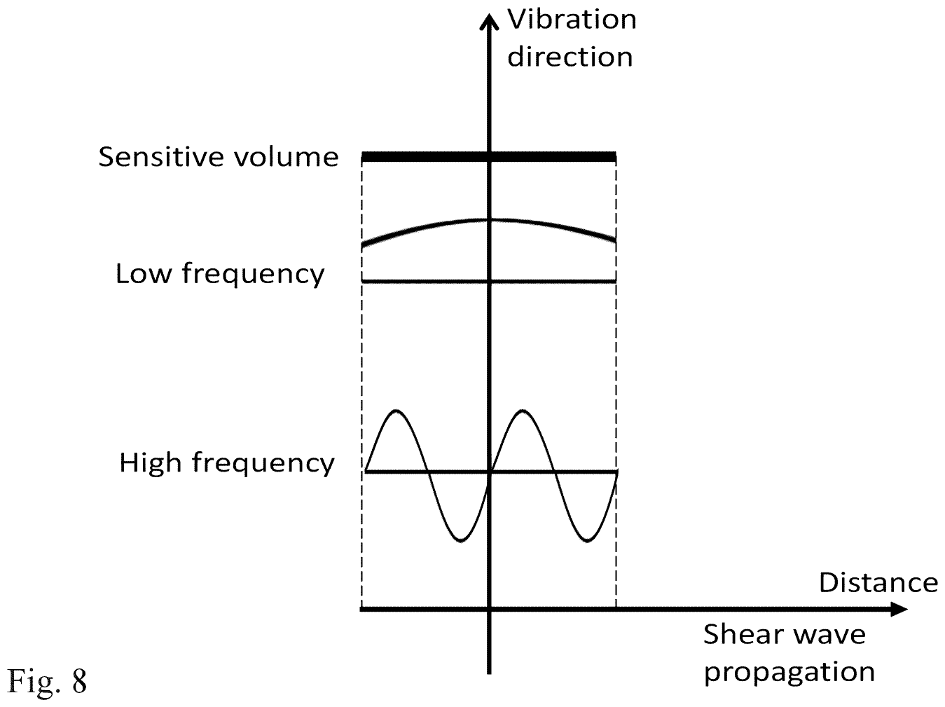

The disclosed POC MRE approach may not generate an image. Therefore, the wavelength of the shear wave may be computed by accounting for the extension of the sensitive volume. For example, FIG. 8 illustrates example shear waves with long and short wavelengths relative to the size of the sensitive volume. The limited extension of the sensitive volume can have an impact on the phase of the NMR signal, which is particularly noticeable when the wavelength is of the order of the size of the sensitive volume.

As the distance to the vibration source increases, the amplitude of the shear wave may generally diminish. This effect is less pronounced at the low vibration frequencies of POC MRE, as the attenuation decreases at lower frequencies.

### Characterizing Shear Waves With Spin Echoes

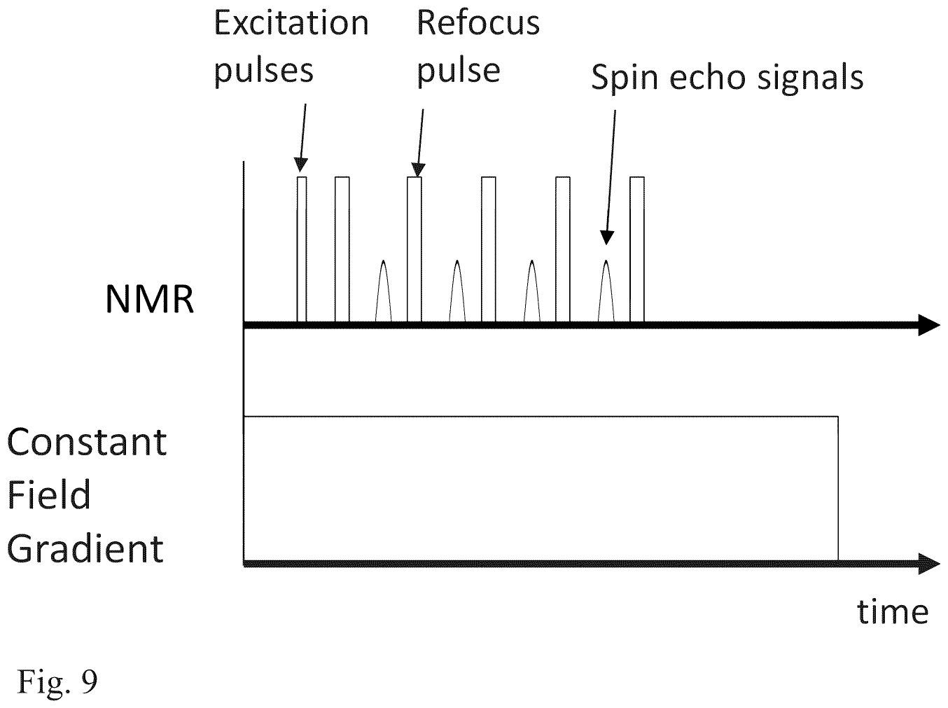

In open NMR systems, the NMR signals are typically spin echoes, as the signals from the free induction decay rapidly—within microseconds—making it difficult to be measured. A commonly used pulse sequence for this purpose is the Carr-Purcell-Meiboom-Gill (CPMG) sequence, where an initial excitation pulse is followed by a series of spin refocusing pulses. The spin echoes are generated between the refocusing pulses. A receiver coil is used to collect the spin echoes—single echo in the case of a Hahn echo or multiple echoes in the case of a CPMG sequence. An example sequence is depicted in FIG. 9. In open NMR systems, the duration of RF pulses is typically on the order of tens of microseconds, while the delay between refocusing pulses is in the range of hundreds of microseconds.

In POC NMR an example shear wave may have an associated tissue displacement of 10 microns. For a gradient G=2 T/m, this displacement means that the spins see a frequency span of about 1 kHz around the Larmor or resonance frequency. To observe changes in this range, a method may be to use longer RF pulses, around 1 ms, therefore the excitation bandwidth may be similar to the displacement associated with the shear wave displacements. Shaped pulses may also be used to limit the bandwidth associated with the pulses. The use of long and shaped pulses are of common use in NMR, but not for the methods presented herein.

Long echo times can introduce more sensitivity to movement: self-diffusion and displacement. These contributions can be separated by measuring the NMR signals with and without external vibrations.

To avoid body motion effects, there is an advantage to use short CPMG trains, or even a single echo to determine the phase shift introduced by the shear waves.

Starting from low frequencies and performing tests at increasing frequency levels, the first null shift of the NMR signals can occurs at f1=Vs/D, where Vs represents the velocity of the shear wave propagating through the sensitive volume of the POC MRE. The amplitude of the excitation vibration can influence the modulation of the NMR signal's phase shift. By identifying the null shifts as the frequency is varied, it becomes possible to determine the wave velocity independently of the vibration amplitude.

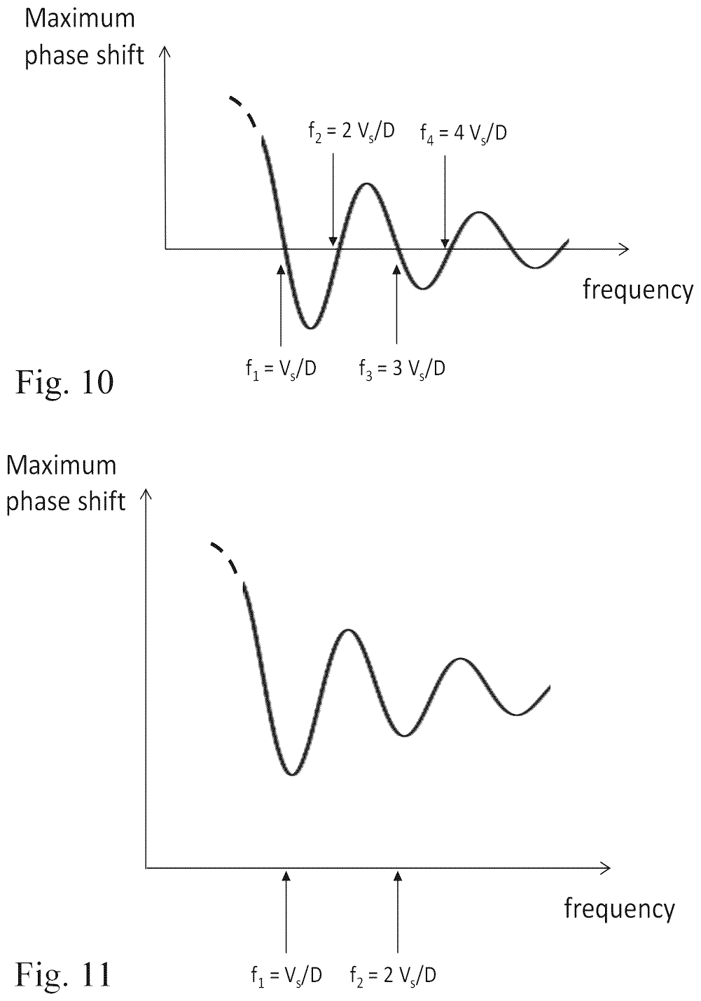

The phase shift nulls may align with multiples of the initial null frequency, as illustrated in FIG. 10. The level of the phase shift relies on the amplitude of the excitation vibration, which typically varies with frequency. This is not relevant when the null points are used for the wave velocity or wavelength computation. In this method, the frequencies that result in null phase shifts, regardless of the dephasing between excitation and data collection timing, can be expressed as: fn=nVs/D, where n represents an integer. Once a null phase shift is identified, searching for the next null and calculating the frequency difference enables the determination of the shear wave velocity, given the known dimension of the sensitive volume (D): Vs=ΔfD, for any consecutive frequency null. Therefore, this measurement does not need to be performed at the lowest frequency null point of the phase shift (f1). In the example of a sensitive volume extending 10 cm and shear wave traveling at 2 m/s, the first null is at f1=Δf=20 Hz.

The regions closest to the vibration source may produce the largest phase shift in the NMR signal. An amplitude damping effect may introduce a modulation that impacts the efficacy of the phase shift nulling. To account for this damping effect, a correction factor may be applied, assuming a damping characteristic for the liver tissue at the operating frequency. It is important to note that the damping effect may be less pronounced or even insignificant at lower frequencies.

If the vibrational excitation transfers into the body more than one mode, interference effects may be observed between the various waves. This may result in no frequencies with null phase shift in the NMR signal. In this case, the estimated frequency span used to calculate the wave speed may be based in the local minima, as shown in FIG. 11.

### T1 Rho Methods—With and Without Vibrations

T1 rho (or T1ρ) relaxation time may describe spin-lattice relaxation in the rotation frame at the presence of an external RF pulse in the transverse plane. T1ρ relaxation may be sensitive to the low-frequency motional processes around or at spin-lock frequency. Areas with increased fibrosis typically exhibit prolonged T1ρ relaxation times compared to healthy liver tissue. Therefore, T1ρ MRI can provide a non-invasive means to assess the degree of fibrosis and potentially differentiate between various stages of liver disease.

In liver studies, T1ρ relaxation is a technique used to assess the health and condition of the liver tissue. It can provide insights into the biochemical and structural properties of the liver, particularly related to fibrosis, which is the excessive accumulation of scar tissue.

The T1ρ relaxation times may reflect the mobility and interaction of water molecules and macromolecules, such as collagen, within the liver tissue. Fibrosis involves the accumulation of collagen fibers in the liver as a response to chronic liver injury, such as that caused by viral hepatitis, alcohol abuse, or non-alcoholic fatty liver disease.

T1ρ relaxation time provides a new mechanism that differs from T1- and T2-weighted NMR responses, and is useful to study low-frequency motional processes and chemical exchange in biological tissues. T1ρ at low spin-lock frequency, may be sensitive to B0 and B1 inhomogeneity.

T1ρ is proposed here as a method to enhance the sensitivity in POC MRE—i.e. open NMR with external controlled vibrations. This can be implemented in the open NMR probe as a means to provide a fibrosis biomarker. T1ρ can then be used in combination with shear waves to probe the stiffness of a tissue. This is based on the sensitivity of T1ρ to low frequencies (hundreds of Hz to few kHz).

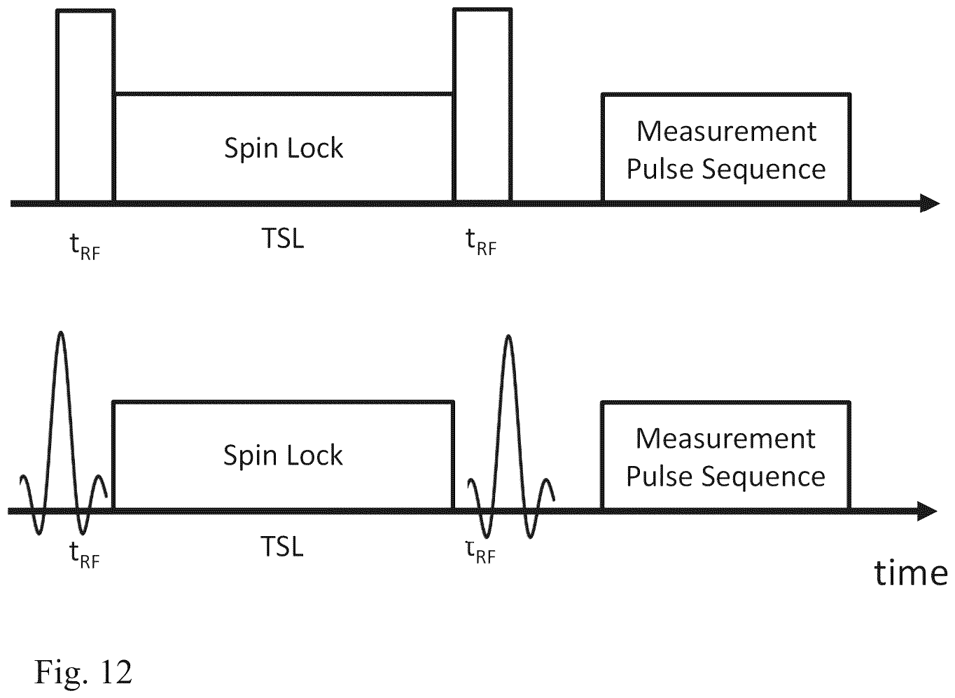

During a T1ρ scan, a spin-lock pulse may be applied to the liver tissue. This spin-lock pulse cam be an additional radiofrequency (RF) pulse that causes the nuclear spins in the tissue to precess around an axis perpendicular to the main magnetic field (B0). By applying the spin-lock pulse at a frequency slightly offset from the Larmor frequency, the exchange of energy between the liver tissue and its surrounding environment may be affected, leading to changes in the T1ρ relaxation times, as depicted in FIG. 12.

The T1ρ sequence may be used as a set of preparation pulses. In the case of open NMR, this preparation may follow a sequence that generates spin echoes—as a means of measuring NMR signals in the presence of high field gradients. T1ρ relaxation process may maximize at spin lock frequency, which is generally in the low frequency range. Various composite spin-lock pulses have been proposed to alleviate the influence of field inhomogeneity so as to reduce the banding-like spin-lock artifacts, an advantage for open NMR testing.

When shear waves are present, the characterization of waves may be performed using T1ρ. The advantage of this approach is based on the sensitivity of T1ρ to low frequencies. This has two considerations: a) the T1ρ method may be inherently sensitive to low frequencies and b) the T1ρ spin locking pulses are long, reducing the observed frequency bandwidth. This last advantage means that the tissue displacements by the shear waves may be magnified by reducing the thickness of the observable (excited) sensitive volume.

Therefore, a T1ρ preparation preceding—for example—a CPMG pulse sequence provides sensitivity to the shear wave, which has a low frequency associated with it, when compared to the Larmor frequency, typically in MHz with this scheme, the T1ρ may be measured by evaluating the NMR signal recovery, S=So e^{(-TSL/T1ρ)}. With a similar explanation as the one provided in the previous section, the limited extent of the sensitive volume is what allows the computation of the wavelength and velocity of the shear wave. In the case of the spin-locking pulse, vibrations during the long pulse may cause a measurable change in the T1ρ values. In the case of V/D>=1/TSL, the vibration causes a modulation on the response, similar to the behavior described in FIG. 11, which allows computation of the velocity of the shear wave.

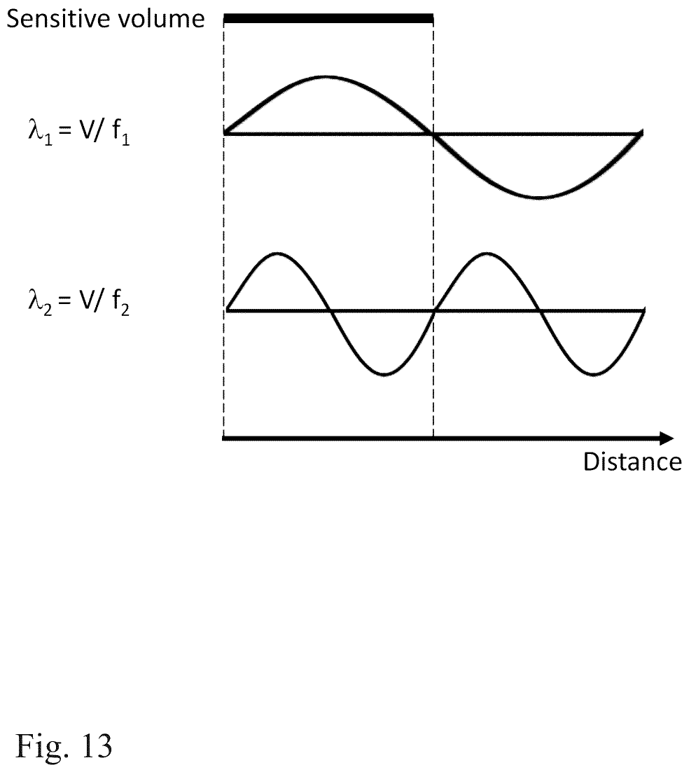

FIG. 13 is a representation of wavelengths in relation to the size of the sensitive volume for two frequencies, according to embodiments of the present disclosure.

### Characterization of Shear Waves With Diffusion Methods

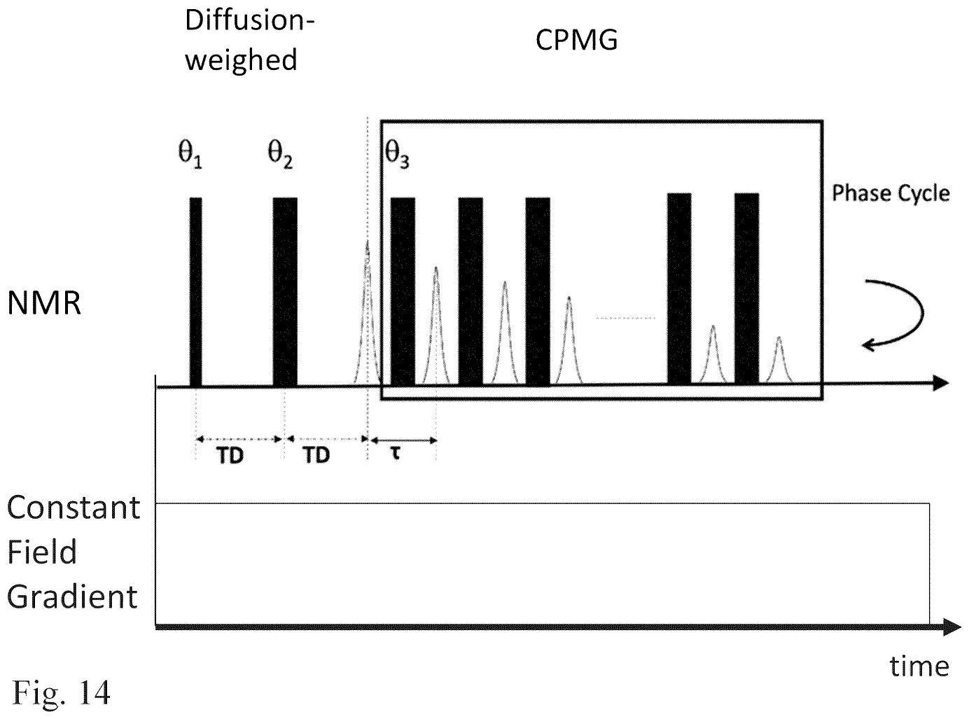

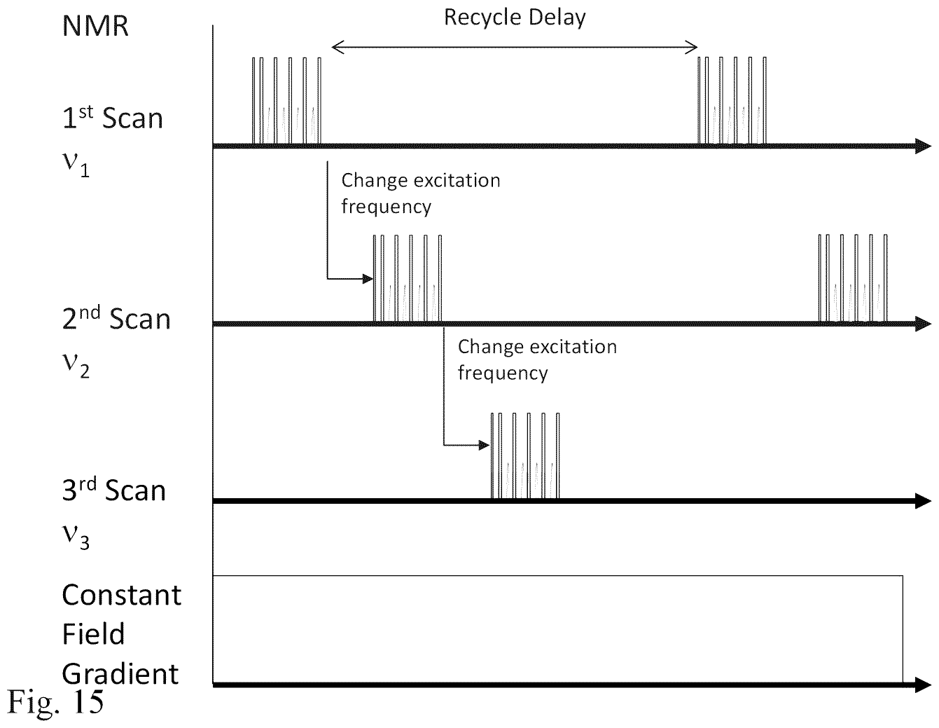

Diffusion-weighed pulse sequences are sensitive to the self-diffusion of protons and to other proton displacements—for example the movement caused by a shear wave. Effects may be separated by using a diffusion-weighed sequence with the vibration on and off. The diffusion-weighed sequence may have a set of preparation pulses following by an NMR reading stage, as shown in FIG. 14.

Such sequences may be sensitive to motion in the range of the diffusion time, TD. In the sequence shown in FIG. 14, the reduction in NMR signal with the shear wave may be dominated by the effect during the preparation pulse. In particular, the diffusion effect may be reduced by using close together refocusing pulses in the CPMG stage. For, f=V/D<1/TD, the displacements may be dominated by diffusion. The reduction in signal amplitude due to diffusion can be measured with no vibration. As the vibration frequency increases, the NMR signal amplitude and phase may be affected by the share wave effect. As explained in the Spin Echoes section, the effect may be modulated by the limited extension of the sensitive volume, D. In the same manner, the shear wave velocity and the wavelength may be computed by looking at the minima in the signal dependency with the vibration frequency. For example, as shown in FIGS. 10-11.

### Sensitivity Enhancement

The NMR measurements in open NMR may be performed in the presence of a high field gradient. The thickness of the sensitive volume associated with the frequency bandwidth of the RF pulses may be narrow. Typically, tens to hundreds of microns. Additionally, in order to allow for magnetization recovery after an RF pulse or train of pulses, there is a recovery time, commonly called recycling delay, before a new scan is performed. During this wait time, usually no scan is performed.

A method to increase sensitivity may increase the number of NMR scans per unit of time. This may be performed by launching one or more additional scans during the wait time of the original scan. If the scans are offset by a frequency larger than the bandwidth of the previous scans, there is no need for an extended recycling delay. For example, as depicted in FIG. 15. In this manner, more scans may be completed per unit of time, allowing for sensitivity increase by signal averaging.

This method may be effective when long or shaped RF excitation and refocusing pulses are used, as this reduces the differences between each of the scans. The concept may be applied to any configuration of pulse sequences, as it's a mean to repeat the processes avoiding spin system saturation.

### Data Collection

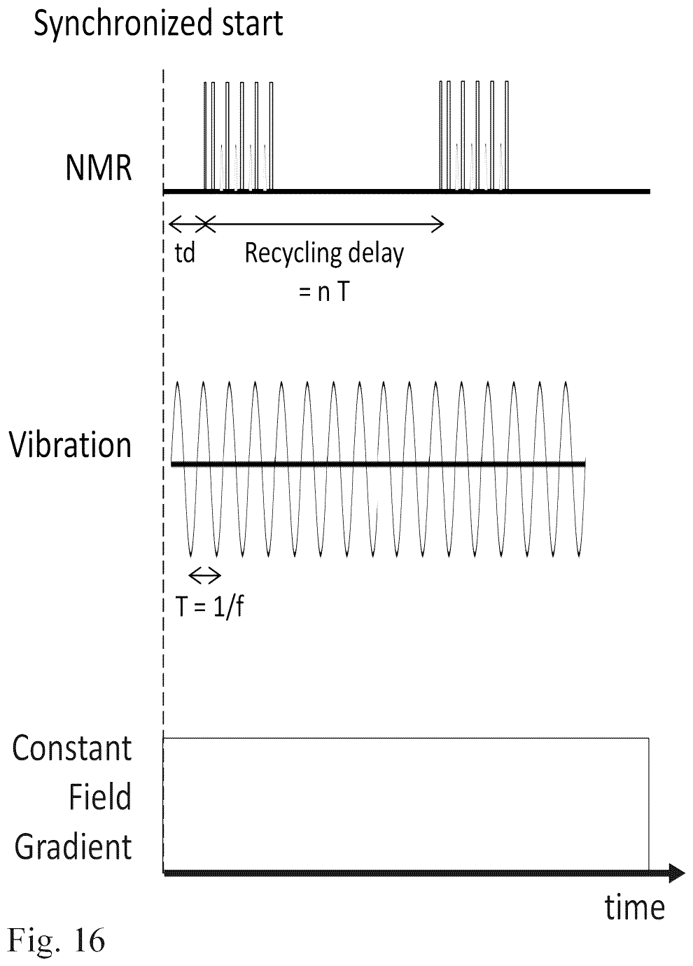

An example of temporal evolution of shear waves at a specific point within the sensitive volume, along with a pulse sequence trigger scheme, is depicted in FIG. 16. While the timing between pulse sequences may be adjusted when performing the test without synchronizing the NMR pulse sequence and the vibration, it may be prudent to maintain sufficient sensitivity to enable accurate signal amplitude and phase shift measurements. To measure the phase shifts and signal amplitude of the complex NMR signals, the NMR test is designed to have a reference phase with no excitation.

When the POC MRE instrument allows for synchronization between the NMR pulses and the actuator, signal averaging may be performed by repeating the tests. And the start point for the NMR pulse sequence (TD in FIG. 16) may be controlled.

The NMR protocol for synchronized POC MRE may include:
(1) Selecting a low frequency based on the range of wave velocities to be measured, e.g. 10 Hz.
(2) Setting a delay for the pulse sequence, TD.
(3) Starting the actuator and then the timed NMR pulse sequence.
(4) Repeating the acquisition protocol and signal average if it is necessary to increase sensitivity.
(5) Repeating for various TD values.
(6) Finding the TD for maximum and/or minimum phase shift or signal amplitude in the NMR signal.
(7) Increasing the frequency and repeat the entire protocol.

The process may be repeated for several frequencies until a null or local minimum is found. It can be extended to describe de modulation, as shown in FIGS. 10-11.

If the frequency is chosen based on knowledge of the range of shear wave velocities (Vs), it may be adequate to estimate Vs using the first null point (f1) for the phase shift. In this case, there would be little to no difference in the NMR signal phase between excitation and non-excitation readings. Alternatively, if there is a noticeable phase difference, it becomes necessary to increment the frequency until the next null or minimum phase or amplitude point is identified. Vs can then be calculated using the formula: Vs=Δf*D, as explained above.

### Example Implementation of Actuator

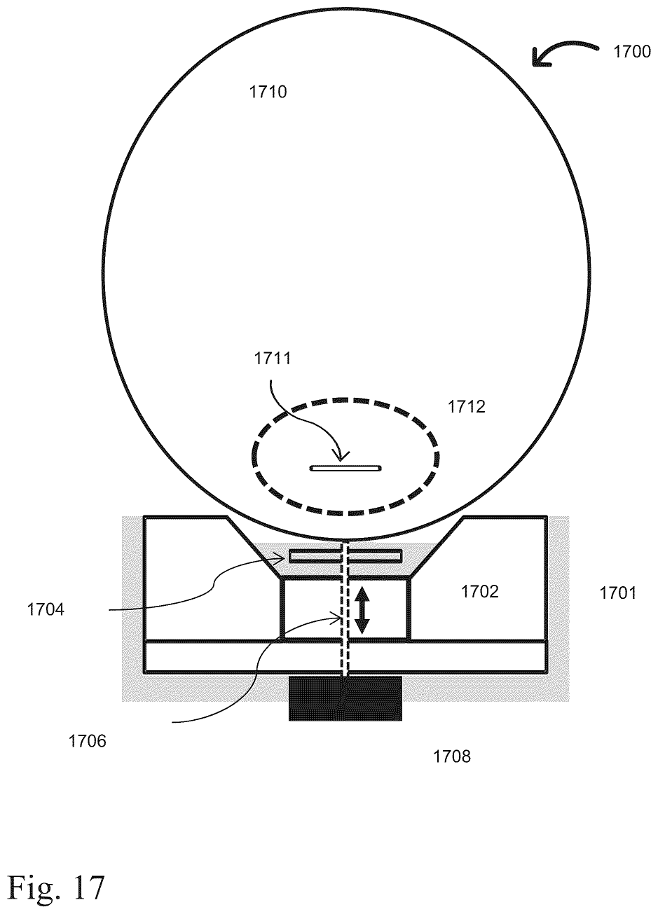

As depicted herein, there are multiple methods for inducing shear waves in the target tissues during POC MRE. It is relevant for the external vibration to generate tissue oscillations aligned with the static field gradient. An efficient POC MRE device can incorporate an actuator positioned in regions of low static field, utilizing a solid rod to transmit the vibration, as illustrated in FIG. 17. In such an example, the POC MRE device 1700 may include an Open NMR probe 1701, with a magnet 1702 and an antenna 1704. The POC MRE may further include a rigid bar or vibration rod 1706 that may transfer the vibrations from the actuator 1708 to the surface of the skin on the body 1710. These vibrations travel into the body 1710 and generate transverse propagation shear waves across the sensitive volume 1711 (within the organ 1712) of the open NMR device 1701.

For actuators that are synchronized with the pulse sequence, the timing control may be directed by the computer or the RF controller components. Alternatively, the actuator may have a timer that triggers the NMR pulses.

While various embodiments of the present disclosure have been described above, it should be understood that they have been presented by way of example only, and not of limitation. Likewise, the various diagrams may depict an example architectural or other configuration for the disclosure, which is done to aid in understanding the features and functionality that can be included in the disclosure. The disclosure is not restricted to the illustrated example architectures or configurations, but the desired features can be implemented using a variety of alternative architectures and configurations. Indeed, it will be apparent to one of skill in the art how alternative functional, logical, or physical partitioning and configurations can be implemented to implement the desired features of the present disclosure. Also, a multitude of different constituent module names other than those depicted herein can be applied to the various partitions. Additionally, with regard to diagrams, operational descriptions, and method claims, the order in which the steps are presented herein shall not mandate that various embodiments be implemented to perform the recited functionality in the same order unless the context dictates otherwise.

Although the disclosure is described above in terms of various embodiments and implementations, it should be understood that the various features, aspects, and functionality described in one or more of the individual embodiments are not limited in their applicability to the particular embodiment with which they are described, but instead can be applied, alone or in various combinations, to one or more of the other embodiments of the disclosure, whether or not such embodiments are described and whether or not such features are presented as being a part of a described embodiment. Thus, the breadth and scope of the present disclosure should not be limited by any of the above-described embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “typical,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “element” does not imply that the components or functionality described or claimed as part of the element are all configured in a common package. Indeed, any or all of the various components of an element can be combined in a single package or separately maintained and can further be distributed in multiple groupings or packages or across multiple locations.

Additionally, the various embodiments set forth herein are described in terms of exemplary diagrams, flow charts, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A nuclear magnetic resonance (NMR) apparatus for elastography using a constant magnetic field gradient, comprising:
a magnetic assembly configured to generate a constant magnetic field gradient within a sample;
a radiofrequency (RF) coil assembly configured to emit RF signals into the sample and receive NMR signals from the sample;
a data acquisition unit connected to the RF coil assembly and configured to acquire NMR signals from the sample during elastography measurement;
a processing unit connected to the data acquisition unit and configured to process the acquired NMR signals to generate elastography data based on the constant magnetic field gradient; and
a display unit connected to the processing unit and configured to display generated elastography results.

2. The NMR apparatus of claim 1, further comprising:
a single-sided magnet configured to generate the constant magnetic field gradient within the sample; and
an antenna connected to the RF coil assembly and configured to emit RF signals into the sample and receive NMR signals from the sample.

3. The NMR apparatus of claim 1, further comprising:
an actuator configured to transfer vibrations to a body under investigation, the actuator comprising a rigid bar; and
a control unit connected to the actuator and configured to control the transfer of vibrations to the body during an elastography test.

4. The NMR apparatus of claim 1, wherein a compact NMR device is mounted on a bed or stand.

5. The NMR apparatus of claim 1, further comprising:
an external actuator positioned in proximity to an NMR probe, said external actuator being configured to transfer vibrations to a body under investigation.

6. The NMR apparatus of claim 1, further comprising:
an external actuator positioned above a body placed on an NMR probe, said external actuator being configured to transfer vibrations to the body for elastography measurements.

7. The NMR apparatus of claim 1, further comprising:
an external actuator positioned on a side of a body placed on an NMR probe, said external actuator being configured to transfer vibrations to the body for elastography measurement.

8. A method for elastography using an NMR apparatus comprising:
generating a constant magnetic field gradient within a sample using a magnetic assembly;
emitting RF signals into the sample and receiving NMR signals from the sample using an RF coil assembly;
acquiring NMR signals from the sample during elastography measurements using a data acquisition unit;
processing the acquired NMR signals using a processing unit to generate elastography signals based on the constant magnetic field gradient; and
displaying generated elastography results using the display unit.

9. The method of claim 8, further comprising:
generating the constant magnetic field gradient within the sample using a single-sided magnet; and
emitting RF signals into the sample and receiving NMR signals from the sample using an antenna.

10. The method of claim 8, further comprising:
transferring vibrations to a body under investigation using an actuator comprising a rigid bar, wherein the actuator is controlled by a control unit.

11. The method of claim 8 wherein a velocity of a wavelength of a shear wave is computed by an effect of an NMR signal phase shifts after a vibration is applied to a body.

12. The method of claim 11, wherein the velocity of the wavelength of the shear wave is computed by finding frequencies with local minima in a phase variation of the NMR signals.

13. The method of claim 12, wherein the velocity of the wavelength of the shear wave is computed by finding the frequencies with local minima in the phase shift of the NMR signals and estimating the wave velocity or wavelength by looking at a difference in the minima.

14. The method of claim 8, further comprising:
utilizing an external actuator positioned in proximity to an NMR probe to transfer vibrations to a body under investigation.

15. The method of claim 8, further comprising:
utilizing an external actuator positioned above a body placed on an NMR probe to transfer vibrations to the body for elastography measurements.

16. The method of claim 8, further comprising:
utilizing an external actuator positioned on a side of a body placed on an NMR probe to transfer vibrations to the body for elastography measurement.

17. The method of claim 8 wherein a stiffness determination does not depend on a T2* measurement of the NMR signal.

18. The method of claim 8, wherein a series of scans are performed after short time delays and by shifting excitation frequency to avoid saturation effects, increasing a number of scans per unit of time to increase sensitivity.

19. The method of claim 8, wherein measuring T1ρ is deterministic of a degree of fibrosis in a body under investigation.

20. The method of claim 19, wherein measuring T1ρ comprises evaluating an NMR signal recovery as a relationship: S=So e^{(-TSL/T1ρ)}.
